SEQUENCE LISTING (33 sequences)

SEQ ID NO: 1 - protein, 5 aa, Artificial Sequence
  linker sequence
GGGGS

SEQ ID NO: 2 - DNA, 39 bp, Artificial Sequence
  linker sequence
ggaggaggaggacggaggaggaggacggaggaggaggac

SEQ ID NO: 3 - DNA, 42 bp, Artificial Sequence
  linker sequence
ggggggggggggccggggggggggggccggggggggggggcc

SEQ ID NO: 4 - DNA, 42 bp, Artificial Sequence
  linker sequence
ggagggggagggcaggagggggagggcaggagggggagggca

SEQ ID NO: 5 - DNA, 41 bp, Artificial Sequence
  linker sequence
ggaggaggaggacggggggggggggcgggaggaggaggaca

SEQ ID NO: 6 - DNA, 98 bp, Artificial Sequence
  linker sequence
ggaggaggaggaagggaggaggaggaagggaggaggaggaagggaggaggaggaagggaggaggaggaagggaggaggaggaagggaggaggaggaag

SEQ ID NO: 7 - DNA, 98 bp, Artificial Sequence
  linker sequence
ggggggggggggagggggggggggggagggggggggggggagggggggggggggagggggggggggggagggggggggggggagggggggggggggag

SEQ ID NO: 8 - DNA, 105 bp, Artificial Sequence
  linker sequence
ggagggggagggagcggagggggagggagcggagggggagggagcggagggggagggagcggagggggagggagcggagggggagggagcggagggggagggagc

SEQ ID NO: 9 - DNA, 97 bp, Artificial Sequence
  linker sequence
ggaggaggaggacggggggggggggccggaggaggaggacaggggggggggggcgggaggaggaggaagggggggggggggagcggaggaggaggac

SEQ ID NO: 10 - protein, 9 aa, Artificial Sequence
  peptide sequence
VVQPGGSLR

SEQ ID NO: 11 - DNA, 27 bp, Artificial Sequence
  peptide sequence
gtggtgcagcctgggggctctctgaga

SEQ ID NO: 12 - protein, 35 aa, Artificial Sequence
  linker sequence
GGGGSGGGGSGGGGSGGGGSGGGGSGGGGSGGGGS

SEQ ID NO: 13 - DNA, 105 bp, Artificial Sequence
  linker sequence
ggaggcggtgggtcaggtggcggaggcagcggtggaggaggtagtggcggtggcggtagtgggggtggaggcagcggaggcggaggcagtgggggcggtggatca

SEQ ID NO: 14 - DNA, 105 bp, Artificial Sequence
  linker sequence
ggaggcggtgggtcaggtggcggaggcagcggtggaggaggtagtggcggtggcggtagtgggggtggaggcagcggaggcggaggcagtgggggcggtggatcc

SEQ ID NO: 15 - DNA, 105 bp, Artificial Sequence
  linker sequence
ggaggcggtgggtcaggtggcggaggcagcggtggaggaggtagtggcggtggcggtagtgggggtggaggcagcggaggcggaggcagtgggggcggtggctca

SEQ ID NO: 16 - protein, 20 aa, Artificial Sequence
  peptide fragment
YWGQGTLVTVSSGGGGSGGG

SEQ ID NO: 17 - protein, 46 aa, Artificial Sequence
  peptide fragment
DSGGGGSGGGGSGGGGSGGGGSGGGGSEVQLVESGGGVVQPGGSLR

SEQ ID NO: 18 - protein, 18 aa, Artificial Sequence
  peptide fragment
YWGQGTLVTVSSGGGGSG

SEQ ID NO: 19 - protein, 48 aa, Artificial Sequence
  peptide fragment
DGGSGGGGSGGGGSGGGGSGGGGSGGGGSEVQLVESGGGVVQPGGSLR

SEQ ID NO: 20 - protein, 29 aa, Artificial Sequence
  peptide fragment
YWGQGTLVTVSSGGGGSGGGGSGGGGSGG

SEQ ID NO: 21 - protein, 37 aa, Artificial Sequence
  peptide fragment
DGSGGGGSGGGGSGGGGSEVQLVESGGGVVQPGGSLR

SEQ ID NO: 22 - protein, 35 aa, Artificial Sequence
  peptide fragment
YWGQGTLVTVSSGGGGSGGGGSGGGGSGGGGSGGG

SEQ ID NO: 23 - protein, 31 aa, Artificial Sequence
  peptide fragment
DSGGGGSGGGGSEVQLVESGGGVVQPGGSLR

SEQ ID NO: 24 - protein, 38 aa, Artificial Sequence
  peptide fragment
YWGQGTLVTVSSGGGGSGGGGSGGGGSGGGGSGGGGSG

SEQ ID NO: 25 - protein, 28 aa, Artificial Sequence
  peptide fragment
DGGSGGGGSEVQLVESGGGVVQPGGSLR

SEQ ID NO: 26 - protein, 13 aa, Artificial Sequence
  peptide fragment
YWGQGTLVTVSSG

SEQ ID NO: 27 - protein, 53 aa, Artificial Sequence
  peptide fragment
DGGSGGGGSGGGGSGGGGSGGGGSGGGGSGGGGSEVQLVESGGGVVQPGGSLR

SEQ ID NO: 28 - protein, 27 aa, Artificial Sequence
  peptide fragment
YWGQGTLVTVSSGGGGSGGGGSGGGGS

SEQ ID NO: 29 - protein, 39 aa, Artificial Sequence
  peptide fragment
DGGGSGGGGSGGGGSGGGGSEVQLVESGGGVVQPGGSLR

SEQ ID NO: 30 - protein, 40 aa, Artificial Sequence
  peptide fragment
YWGQGTLVTVSSGGGGSGGGGSGGGGSGGGGSGGGGSGGG

SEQ ID NO: 31 - protein, 26 aa, Artificial Sequence
  peptide fragment
DSGGGGSEVQLVESGGGVVQPGGSLR

SEQ ID NO: 32 - protein, 43 aa, Artificial Sequence
  peptide fragment
YWGQGTLVTVSSGGGGSGGGGSGGGGSGGGGSGGGGSGGGGSG

SEQ ID NO: 33 - protein, 23 aa, Artificial Sequence
  peptide fragment
DGGSEVQLVESGGGVVQPGGSLR

(19) United States
(12) Patent Application Publication
(10) Pub. No.: US 2020/0392512 A1
(43) Pub. Date: Dec. 17, 2020

(54) NUCLEOTIDE SEQUENCES ENCODING PEPTIDE LINKERS

(71) Applicant: Ablynx N.V., Ghent-Zwijnaarde (BE)

(72) Inventors: Veronique De Brabandere, Gent (BE); Ann Brige, Ertvelde (BE); Patrick Stanssens, Nazareth (BE); Pieter-Jan De Bock, Gentbrugge (BE); Tom Merchiers, Oudenaarde (BE); Antonin De Fougerolles, Oxford (GB)

(73) Assignee: Ablynx N.V., Ghent-Zwijnaarde (BE)

(21) Appl. No.: 16/975,422

(22) PCT Filed: Feb. 26, 2019
(86) PCT No.: PCT/EP2019/054697

§ 371 Date: Aug. 25, 2020

Related U.S. Application Data
(60) Provisional application No. 62/634,985, filed on Feb. 26, 2018.

(51) Int. Cl. C12N 15/62; C07K 16/46

(52) U.S. Cl. CPC C12N 15/62; C07K 16/468; C07K 2317/569; C07K 2317/35; C07K 2319/00

(57) ABSTRACT

The invention provides improved nucleotide sequences and nucleic acids that encode glycine serine linkers and that use an excess of GGA, GGG, and GGT/GGU codons to encode the glycine residues. The invention further relates to nucleotide sequences and nucleic acids that encode (fusion) proteins and polypeptides comprising glycine serine linkers, which nucleotide sequences and nucleic acids comprise such improved nucleotide sequences and nucleic acids of the invention.

The present invention relates to improved nucleotide sequences and nucleic acids that encode peptide linkers.

The present invention also relates to nucleotide sequences and nucleic acids that encode (fusion) proteins and polypeptides that contain peptide linkers, which nucleotide sequences and nucleic acids contain such improved nucleotide sequences and nucleic acids that encode peptide linkers.

The present invention also relates to methods for expressing/producing (fusion) proteins and polypeptides containing peptide linkers, which involve the use of such improved nucleotide sequences and nucleic acids that encode peptide linkers.

Other aspects, embodiments, uses and advantages of the present invention will become clear from the further description herein.

The use of peptide linkers to link two or more proteins, peptides, peptide moieties, binding domains or binding units is well known in the art. One often used class of peptide linker are known as the “Gly-Ser” or “GS” linkers. These are linkers that essentially consist of glycine (G) and serine (S) residues, and usually comprise one or more repeats of a peptide motif such as the GGGGS motif (for example, have the formula (Gly-Gly-Gly-Gly-Ser)ₙ in which n may be 1, 2, 3, 4, 5, 6, 7 or more). Some often used examples of such GS linkers are 15GS linkers (n=3) and 35GS linkers (n=7). Reference is for example made to Chen et al., Adv. Drug Deliv. Rev. 2013 Oct. 15; 65(10): 1357-1369; and Klein et al., Protein Eng. Des. Sel. (2014) 27 (10): 325-330.

Polypeptides and (fusion) proteins that comprise such GS linkers are often produced by suitably expressing a genetic construct that comprises two or more nucleotide sequences encoding the relevant peptide moieties to be linked, in which these nucleotide sequences encoding the peptide moieties are suitably and operably linked via one or more nucleotide sequences that encode the one or more GS linker(s), such that upon suitable expression in a suitable host cell or host organism, the desired fusion protein or polypeptide is obtained, optionally after suitable steps for isolation and/or purification. Some preferred, but non-limiting examples of such genetic constructs (using Nanobodies as representative examples of the peptides to be linked, see the legend to Table III) are shown schematically in FIG. 1, in which NB₁, NB₂, NB_{A}, NB_{B}, etc. indicate nucleotide sequences that encode the peptide moieties to be linked, and L₁, L₂, L₃, etc. indicate nucleotide sequences that encode a suitable GS linker. Such genetic constructs may be DNA or RNA, and may for example be in the form of a suitable vector, such as an expression vector. All of this is well-known in the art of protein engineering; reference is for example made to the standard handbooks, such as Sambrook et al. and Ausubel et al. referred to herein.

It is also generally known that, due to the degeneracy of the genetic code, in the nucleotide sequences that encode GS linkers, each one of four different codons may be used to encode a glycine residue, namely GGU (or GGT), GGC, GGA and/or GGG (it is similarly known that the serine residues in a GS linker may be encoded by an UCU (or TCT), UCC (or TCC), UCA (or TCA), UCG (or TCG), AGU (or AGT) and/or AGC codon.

It has now been found that improved nucleotide sequences encoding GS linkers may be provided by using an excess of GGA and GGG codons to encode the glycine residues in the GS linker (i.e. compared to the amount of GGT/GGU and/or GGC codons).

It has further been found that improved nucleotide sequences encoding GS linkers may be provided by using an excess of GGA, GGG, and GGT/GGU codons to encode the glycine residues in the GS linker (i.e. compared to the amount of GGC codons).

Thus, in a first aspect, the invention relates to a nucleotide sequence and/or a nucleic acid that encodes a GS linker (as further defined herein), in which more than 70%, preferably more than 85%, more preferably more than 90%, such as more than 95% and up to 99% and more (including 100%) of the codons that encode a glycine residue in the GS linker are either GGA, GGG or GGT/GGU.

In this aspect, the invention also relates to a nucleotide sequence and/or a nucleic acid that encodes a GS linker (as further defined herein), in which more than 70%, preferably more than 85%, more preferably more than 90%, such as more than 95% and up to 99% and more (including 100%) of the codons that encode a glycine residue in the GS linker are either GGA or GGG.

In this aspect, the invention also relates to a nucleotide sequence and/or a nucleic acid that encodes a GS linker (as further defined herein), in which less than 30%, preferably less than 15%, more preferably less than 10%, such as less than 5% and up to less than 1% or lower (including 0%) of the codons that encode a glycine residue in the GS linker are GGC.

In a further aspect, the invention relates to a nucleotide sequence and/or a nucleic acid that encodes a peptide linker, in which more than 70%, preferably more than 85%, more preferably more than 90%, such as more than 95% and up to 99% and more (including 100%) of the codons that encode a glycine residue in said peptide linker are either GGA, GGG or GGT/GGU.

In this aspect, the invention also relates to a nucleotide sequence and/or a nucleic acid that encodes a peptide linker (as further described herein), in which the peptide linker encoded by said nucleotide sequence or nucleic acid comprises or essentially consists of glycine and serine residues, in which more than 70%, preferably more than 85%, more preferably more than 90%, such as more than 95% and up to 99% and more (including 100%) of the codons that encode a glycine residue in said peptide linker are either GGA or GGG.

In this aspect, the invention also relates to a nucleotide sequence and/or a nucleic acid that encodes a peptide linker, in which less than 30%, preferably less than 15%, more preferably less than 10%, such as less than 5% and up to less than 1% or lower (including 0%) of the codons that encode a glycine residue in said peptide linker are GGC.

As further described herein, the peptide linkers encoded by said nucleotide sequences or nucleic acids will generally comprise at least 5 amino acid residues and up to 50 amino acid residues or more (but in practice will usually comprise between 10 and 40 amino acid residues, such as about 15 amino acid residues to about 35 amino acid residues). Also, as further described herein, the peptide linkers encoded by said nucleotide sequences or nucleic acids will usually contain an excess of glycine residues compared to the number of serine residues, for example between 3 and 6 glycine residues for each serine residue. Also, often, the peptide linkers encoded by said nucleotide sequences or nucleic acids will contain one or more (such as two or more) repeats of a sequence motif. In a further aspect, the invention relates to a nucleotide sequence and/or a nucleic acid that encodes a peptide linker (as further described herein), in which the peptide linker encoded by said nucleotide sequence or nucleic acid comprises or essentially consists of one or more (such as two or more) repeats of the sequence motif GGGGS (SEQ ID NO:1), in which more than 70%, preferably more than 85%, more preferably more than 90%, such as more than 95% and up to 99% and more (including 100%) of the codons that encode a glycine residue in said peptide linker are either GGA, GGG or GGT/GGU.

In this aspect, the invention also relates to a nucleotide sequence and/or a nucleic acid that encodes a peptide linker (as further described herein), in which the peptide linker encoded by said nucleotide sequence or nucleic acid comprises or essentially consists of one or more (such as two or more) repeats of the sequence motif GGGGS (SEQ ID NO:1), in which more than 70%, preferably more than 85%, more preferably more than 90%, such as more than 95% and up to 99% and more (including 100%) of the codons that encode a glycine residue in said peptide linker are either GGA or GGG.

In this aspect, the invention also relates to a nucleotide sequence and/or a nucleic acid that encodes a peptide linker (as further described herein), in which the peptide linker encoded by said nucleotide sequence or nucleic acid comprises or essentially consists of one or more (such as two or more) repeats of the sequence motif GGGGS (SEQ ID NO:1), in which less than 30%, preferably less than 15%, more preferably less than 10%, such as less than 5% and up to less than 1% or lower (including 0%) of the codons that encode a glycine residue in said peptide linker are GGC.

For example, in this aspect of the invention, the peptide linker encoded by said nucleotide sequence or nucleic acid may comprise or essentially consists of 2, 3, 4, 5, 6, 7, 8, 9 or 10 repeats of the sequence motif GGGGS.

In a further aspect, the invention relates to a nucleotide sequence and/or a nucleic acid that encodes a peptide linker (as further described herein), in which the peptide linker encoded by said nucleotide sequence or nucleic acid is of the formula (Gly-Gly-Gly-Gly-Ser)ₙ (in which n may be 1, 2, 3, 4, 5, 6, 7 or more), in which more than 70%, preferably more than 85%, more preferably more than 90%, such as more than 95% and up to 99% and more (including 100%) of the codons that encode a glycine residue in said peptide linker are either GGA, GGG or GGT/GGU.

In this aspect, the invention also relates to a nucleotide sequence and/or a nucleic acid that encodes a peptide linker (as further described herein), in which the peptide linker encoded by said nucleotide sequence or nucleic acid is of the formula (Gly-Gly-Gly-Gly-Ser)ₙ (in which n may be 1, 2, 3, 4, 5, 6, 7 or more), in which more than 70%, preferably more than 85%, more preferably more than 90%, such as more than 95% and up to 99% and more (including 100%) of the codons that encode a glycine residue in said peptide linker are either GGA or GGG.

In this aspect, the invention also relates to a nucleotide sequence and/or a nucleic acid that encodes a peptide linker (as further described herein), in which the peptide linker encoded by said nucleotide sequence or nucleic acid is of the formula (Gly-Gly-Gly-Gly-Ser)ₙ (in which n may be 1, 2, 3, 4, 5, 6, 7 or more), in which less than 30%, preferably less than 15%, more preferably less than 10%, such as less than 5% and up to less than 1% or lower (including 0%) of the codons that encode a glycine residue in said peptide linker are GGC.

For example, in this aspect of the invention, the peptide linker encoded by said nucleotide sequence or nucleic acid may comprise or essentially consists of 2, 3, 4, 5, 6, 7, 8, 9 or 10 repeats of the sequence motif GGGGS.

In a further aspect, the invention relates to a nucleotide sequence and/or a nucleic acid of the general formula

(Aₓ-Bₚ-A_{y}-B_{q})ₙ,

in which:
A represents a codon encoding a glycine residue which may independently be (chosen from) a GGU (or GGT), GGC, GGA and/or GGG codon; and
B represents a codon encoding a serine residue which may independently be (chosen from) a UCU (or TCT), UCC (or TCC), UCA (or TCA), UCG (or TCG), AGU (or AGT) and/or AGC codon;
x is an integer from 0 to 10 (and preferably from 0 to 5), and y is an integer from 0 to 10 (and preferably 0 to 5), such that the sum of (x+y) is between 1 and 10, and preferably 3, 4, 5, 6, 7 or 8;
p is 0 or 1, and q is 0 or 1, such that the sum of (p+q) is 2 or 1 and is preferably 1;
n is an integer from 1 to 10 (i.e. such that the nucleotide sequence and/or a nucleic acid comprises n repeats of the motif (Aₓ-Bₚ-A_{y}-B_{q}) in which A, B, p, q, x and y are as described herein);
in each repeat of motif (Aₓ-Bₚ-A_{y}-B_{q}), each A, B, p, q, x and y may independently be as described herein (but according to a preferred aspect, in each repeat of the motif (Aₓ-Bₚ-A_{y}-B_{q}), each A, B, p, q, x and y are the same);
   provided that more than 70%, preferably more than 85%, more preferably more than 90%, such as more than 95% and up to 99% and more (including 100%) of the codons that encode a glycine residue (as represented by A in the formulas of Table I) are either GGA, GGG or GGT/GGU;
   provided that more than 70%, preferably more than 85%, more preferably more than 90%, such as more than 95% and up to 99% and more (including 100%) of the codons that encode a glycine residue (as represented by A in the formulas of Table I) are either GGA or GGG; and/or
   provided that less than 30%, preferably less than 15%, more preferably less than 10%, such as less than 5% and up to less than 1% or lower (including 0%) of the codons that encode a glycine residue (as represented by A in the formulas of Table I) are GGC.

In a further aspect, the invention relates to a nucleotide sequence and/or a nucleic acid of the general formula

(Aₓ-B)ₙ,

in which:
A represents a codon encoding a glycine residue which may independently be (chosen from) a GGU (or GGT), GGC, GGA and/or GGG codon; and
B represents a codon encoding a serine residue which may independently be (chosen from) a UCU (or TCT), UCC (or TCC), UCA (or TCA), UCG (or TCG), AGU (or AGT) and/or AGC codon;
x is an integer from 1 to 10, and is preferably 3, 4, 5, 6, 7 or 8;
n is an integer from 1 to 10 (i.e. such that the nucleotide sequence and/or a nucleic acid comprises n repeats of the motif (Aₓ-B), in which each A, B and x are as described herein);
in each repeat of motif (Aₓ-B), each A, B and x may independently be as described herein (but according to a preferred aspect, in each repeat of the motif (Aₓ-B), each A, B and x are the same);
   provided that more than 70%, preferably more than 85%, more preferably more than 90%, such as more than 95% and up to 99% and more (including 100%) of the codons that encode a glycine residue (as represented by A in the formulas of Table I) are either GGA, GGG, or GGT/GGU;
   provided that more than 70%, preferably more than 85%, more preferably more than 90%, such as more than 95% and up to 99% and more (including 100%) of the codons that encode a glycine residue (as represented by A in the formulas of Table I) are either GGA or GGG; and/or
   provided that less than 30%, preferably less than 15%, more preferably less than 10%, such as less than 5% and up to less than 1% or lower (including 0%) of the codons that encode a glycine residue (as represented by A in the formulas of Table I) are GGC.

In a further aspect, the invention relates to a nucleotide sequence and/or a nucleic acid of one of the formulas shown in Table I, in which:
A represents a codon encoding a glycine residue which may independently be (chosen from) a GGU (or GGT), GGC, GGA and/or GGG codon; and
B represents a codon encoding a serine residue which may independently be (chosen from) a UCU (or TCT), UCC (or TCC), UCA (or TCA), UCG (or TCG), AGU (or AGT) and/or AGC codon;
   provided that more than 70%, preferably more than 85%, more preferably more than 90%, such as more than 95% and up to 99% and more (including 100%) of the codons that encode a glycine residue (as represented by A in the formulas of Table I) are either GGA, GGG, or GGT/GGU;
   provided that more than 70%, preferably more than 85%, more preferably more than 90%, such as more than 95% and up to 99% and more (including 100%) of the codons that encode a glycine residue (as represented by A in the formulas of Table I) are either GGA or GGG; and/or
   provided that less than 30%, preferably less than 15%, more preferably less than 10%, such as less than 5% and up to less than 1% or lower (including 0%) of the codons that encode a glycine residue (as represented by A in the formulas of Table I) are GGC.

Generally, the nucleotide sequences and nucleic acids described herein which encode Gly-Ser linkers and in which the glycine residues in said GS linkers are predominantly or exclusively encoded by GGA, GGG, or GGT/GGU codons are also referred to herein as “GS linker-encoding sequence(s) of the invention”. Generally, the nucleotide sequences and nucleic acids described herein which encode Gly-Ser linkers and in which the glycine residues in said GS linkers are predominantly or exclusively encoded by GGA or GGG codons are also referred to herein as “GS linker-encoding sequence(s) of the invention”. Generally, the nucleotide sequences and nucleic acids described herein which encode Gly-Ser linkers and in which almost none or not any of the glycine residues in said GS linkers are encoded by GGC codons are also referred to herein as “GS linker-encoding sequence(s) of the invention”.

In one preferred but non-limiting aspect of the invention, more than 95%, and up to 99% or more (and including 100%) of the codons that encode a glycine residue in a GS linker-encoding sequence of the invention are either GGA, GGG, or GGT/GGU.

In one preferred but non-limiting aspect of the invention, more than 95%, and up to 99% or more (and including 100%) of the codons that encode a glycine residue in a GS linker-encoding sequence of the invention are either GGA or GGG.

In one preferred but non-limiting aspect of the invention, less than 5%, and up to less than 1% or lower (and including 0%) of the codons that encode a glycine residue in a GS linker-encoding sequence of the invention are GGC. Table II gives some representative, but non-limiting, examples of GS linker-encoding sequence(s) of the invention. Other examples of GS linker-encoding sequence(s) of the invention will be clear to the skilled person based on the disclosure herein.

TABLE I
A-A-A-A-B
A-A-A-A-B-A-A-A-A-B
A-A-A-A-B-A-A-A-A-B-A-A-A-A-B
A-A-A-A-B-A-A-A-A-B-A-A-A-A-B-A-A-A-A-B
A-A-A-A-B-A-A-A-A-B-A-A-A-A-B-A-A-A-A-B-A-A-A-A-B
A-A-A-A-B-A-A-A-A-B-A-A-A-A-B-A-A-A-A-B-A-A-A-A-B-A-A-A-A-B
A-A-A-A-B-A-A-A-A-B-A-A-A-A-B-A-A-A-A-B-A-A-A-A-B-A-A-A-A-B-A-A-A-A-B-A-A-A-A-B-A-A-A-A-B
A-A-A-A-B-A-A-A-A-B-A-A-A-A-B-A-A-A-A-B-A-A-A-A-B-A-A-A-A-B-A-A-A-A-B-A-A-A-A-B-A-A-A-A-B-A-A-A-A-B
In the above formula's:
A represents a codon encoding a glycine residue which may independently be (chosen from) a GGU (or GGT), GGC, GGA and/or GGG codon; and
B represents a codon encoding a serine residue which may independently be (chosen from) a UCU, UCC, UCA, UCG, AGU and/or AGC codon.
In the invention, more than 70%, preferably more than 85%, more preferably more than 90%, such as more than 95% and up to 99% and more (including 100%) of the codons that encode a glycine residue (as represented by A in the above formulas) are either GGA, GGG, or GGT/GGU; more than 70%, preferably more than 85%, more preferably more than 90%, such as more than 95% and up to 99% and more (including 100%) of the codons that encode a glycine residue (as represented by A in the above formulas) are either GGA or GGG; and/or less than 30%, preferably less than 15%, more preferably less than 10%, such as less than 5% and up to less than 1% or lower (including 0%) of the codons that encode a glycine residue (as represented by A in the above formulas) are GGC.

TABLE II

| SEQ | | |
|---|---|---|
| ID | GS- | |
| NO | linker | GS linker-encoding sequence(s) of the invention |
| 2 | 15GS | GGAGGAGGAGGAUCUGGAGGAGGAGGAUCUGGAGGAGGAGGA |
| | | UCU |
| 3 | 15GS | GGGGGGGGGGGGUCCGGGGGGGGGGGGUCCGGGGGGGGGGGGU |
| | | CC |
| 4 | 15GS | GGAGGGGGAGGGUCAGGAGGGGGAGGGUCAGGAGGGGGAGGG |
| | | UCA |
| 5 | 15GS | GGAGGAGGAGGAUCUGGGGGGGGGGGGUCGGGAGGAGGAGGA |
| | | UCA |
| 6 | 35GS | GGAGGAGGAGGAAGUGGAGGAGGAGGAAGUGGAGGAGGAGGA |
| | | AGUGGAGGAGGAGGAAGUGGAGGAGGAGGAAGUGGAGGAGGA |
| | | GGAAGUGGAGGAGGAGGAAGU |
| 7 | 35GS | GGGGGGGGGGGGAGUGGGGGGGGGGGGAGUGGGGGGGGGGGG |
| | | AGUGGGGGGGGGGGGAGUGGGGGGGGGGGGAGUGGGGGGGGG |
| | | GGGAGUGGGGGGGGGGGGAGU |
| 8 | 35GS | GGAGGGGGAGGGAGCGGAGGGGGAGGGAGCGGAGGGGGAGGG |
| | | AGCGGAGGGGGAGGGAGCGGAGGGGGAGGGAGCGGAGGGGGA |
| | | GGGAGCGGAGGGGGAGGGAGC |
| 9 | 35GS | GGAGGAGGAGGAUCUGGGGGGGGGGGGUCCGGAGGAGGAGGA |
| | | UCAGGGGGGGGGGGGUCGGGAGGAGGAGGAAGUGGGGGGGGG |
| | | GGGAGCGGAGGAGGAGGAUCU |

Without being limited to any specific explanation, hypothesis or mechanism, it is assumed that the use of such nucleotide sequences (i.e. compared to the use of nucleotide sequences encoding GS linkers that contain a greater amount/proportion of GGU and/or GGC codons; or compared to the use of nucleotide sequences encoding GS linkers that contain a greater amount/proportion of GGC codons) reduces the risk of aspartate residues being erroneously included in the desired GS linkers (instead of the intended glycine residues) and/or reduces the amount of aspartate residues that, upon expression in a suitable host or host organism, are erroneously included in the desired GS linkers.

Thus, when used in the expression and/or production of fusion proteins or polypeptides, the invention also reduces the amount of contaminants that is obtained in the expressed product (i.e. contaminants that contain GS linkers with one or more aspartate residues instead of the intended glycine residues) and also reduces deleterious effects associated with the unwanted presence of aspartate residues in the desired GS linkers, such as undesired isomerization into iso-aspartate, as well as increase susceptibility to proteolytic degradation.

Thus in another aspect, the invention relates to a nucleotide sequence and/or a nucleic acid that encodes a (fusion) protein or fusion polypeptide, in which the fusion protein or polypeptide that is encoded by said nucleotide sequence and/or a nucleic acid comprises two or more peptide moieties that are suitably linked via one or more GS linkers, in which the one or more GS linkers are encoded by one or more GS linker-encoding sequence(s) of the invention (i.e. by a nucleotide sequence or nucleic acid in which more than 70%, preferably more than 85%, more preferably more than 90%, such as more than 95% and up to 99% and more (including 100%) of the codons that encode a glycine residue in the GS linker are either GGG, GGG, or GGT/GGU).

In this aspect, the invention also relates to a nucleotide sequence and/or a nucleic acid that encodes a (fusion) protein or fusion polypeptide, in which the fusion protein or polypeptide that is encoded by said nucleotide sequence and/or a nucleic acid comprises two or more peptide moieties that are suitably linked via one or more GS linkers, in which the one or more GS linkers are encoded by one or more GS linker-encoding sequence(s) of the invention (i.e. by a nucleotide sequence or nucleic acid in which more than 70%, preferably more than 85%, more preferably more than 90%, such as more than 95% and up to 99% and more (including 100%) of the codons that encode a glycine residue in the GS linker are either GGG or GGG).

In this aspect, the invention also relates to a nucleotide sequence and/or a nucleic acid that encodes a (fusion) protein or fusion polypeptide, in which the fusion protein or polypeptide that is encoded by said nucleotide sequence and/or a nucleic acid comprises two or more peptide moieties that are suitably linked via one or more GS linkers, in which the one or more GS linkers are encoded by one or more GS linker-encoding sequence(s) of the invention (i.e. by a nucleotide sequence or nucleic acid in which less than 30%, preferably less than 15%, more preferably less than 10%, such as less than 5% and up to less than 1% or lower (including 0%) of the codons that encode a glycine residue in the GS linker are GGC).

In another aspect, the invention relates to a nucleotide sequence and/or a nucleic acid that encodes a (fusion) protein or fusion polypeptide, in which the fusion protein or polypeptide that is encoded by said nucleotide sequence and/or a nucleic acid comprises two or more peptide moieties that are suitably linked via one or more GS linkers, in which the part(s) of the nucleotide sequence or nucleic acid that encode(s) the GS linker(s) are one or more GS linker-encoding sequence(s) of the invention (i.e. a nucleotide sequences or nucleic acids in which more than 70%, preferably more than 85%, more preferably more than 90%, such as more than 95% and up to 99% and more (including 100%) of the codons that encode a glycine residue in the GS linker are either GGG, GGG, or GGT/GGU).

In this aspect, the invention also relates to a nucleotide sequence and/or a nucleic acid that encodes a (fusion) protein or fusion polypeptide, in which the fusion protein or polypeptide that is encoded by said nucleotide sequence and/or a nucleic acid comprises two or more peptide moieties that are suitably linked via one or more GS linkers, in which the part(s) of the nucleotide sequence or nucleic acid that encode(s) the GS linker(s) are one or more GS linker-encoding sequence(s) of the invention (i.e. a nucleotide sequences or nucleic acids in which more than 70%, preferably more than 85%, more preferably more than 90%, such as more than 95% and up to 99% and more (including 100%) of the codons that encode a glycine residue in the GS linker are either GGG or GGG).

In this aspect, the invention also relates to a nucleotide sequence and/or a nucleic acid that encodes a (fusion) protein or fusion polypeptide, in which the fusion protein or polypeptide that is encoded by said nucleotide sequence and/or a nucleic acid comprises two or more peptide moieties that are suitably linked via one or more GS linkers, in which the part(s) of the nucleotide sequence or nucleic acid that encode(s) the GS linker(s) are one or more GS linker-encoding sequence(s) of the invention (i.e. by a nucleotide sequence or nucleic acid in which less than 30%, preferably less than 15%, more preferably less than 10%, such as less than 5% and up to less than 1% or lower (including 0%) of the codons that encode a glycine residue in the GS linker are GGC).

More generally, in another aspect, the invention relates to a nucleotide sequence or nucleic acid that comprises or contains one or more GS linker-encoding sequence(s) of the invention. Such a nucleotide sequence or nucleic acid is preferably such that, upon expression in a suitable host cell or host organism, it expresses a (fusion) protein or polypeptide that comprises at least one GS linker (i.e. a GS linker encoded by a GS linker-encoding sequence of the invention).

In another aspect, the invention relates to a method for expressing or producing a (fusion) protein or polypeptide, in which said (fusion) protein or polypeptide comprises two or more peptide moieties that are suitably linked via one or more GS linkers, which method comprises suitably expressing, in a suitable host cell or host organism, a nucleotide sequence and/or a nucleic acid encoding said (fusion) protein or polypeptide, in which said nucleotide sequence and/or a nucleic acid comprises or contains one or more GS linker-encoding sequence(s) of the invention (and further is as described herein). Said method may further comprise the optional step of isolating/purifying the (fusion) protein or polypeptide thus expressed.

In another aspect, the invention relates to a host cell or host organism that comprises a nucleotide sequence and/or a nucleic acid that encodes a (fusion) protein or polypeptide that comprises one or more GS linkers, in which said nucleotide sequence, and/or a nucleic acid comprises or contains one or more GS linker-encoding sequence(s) of the invention (and further is as described herein)

In another aspect, the invention relates to a method for expressing or producing a (fusion) protein or polypeptide, in which said (fusion) protein or polypeptide comprises two or more peptide moieties that are suitably linked via one or more GS linkers, which method comprises cultivating a suitable host cell or host organism that comprises a nucleotide sequence and/or nucleic acid that comprises or contains one or more GS linker-encoding sequence(s) of the invention (and that further is as described herein), under conditions such that said host cell or host organism expresses/produces said (fusion) protein or polypeptide (in which said fusion protein or polypeptide comprises one or more GS linkers, i.e. as encoded by the GS linker-encoding sequence(s) of the invention). Said method may further comprise the optional step of isolating/purifying the (fusion) protein or polypeptide thus expressed.

In a further aspect, the invention relates to a (fusion) protein or polypeptide (and in particular, to a (fusion) protein or polypeptide comprising one or more GS linkers) that has been obtained by expression, in a suitable host cell or host organism, of a nucleotide sequence or nucleic acid encoding said (fusion) protein or polypeptide, in which said nucleotide sequence or nucleic acid contains or comprises one or more GS linker-encoding sequence(s) of the invention (and is as further described herein).

In a further aspect, the invention provides a method for reducing the level of Gly to Asp misincorporation in a peptide linker (such as e.g. a GS linker), said method comprising the step of replacing, in the nucleic acid sequence and/or nucleic acid that encodes said peptide linker, at least one GGC codon with a GGG, GGA or GGT/GGU codon.

In this aspect, the invention also provides a method for reducing the level of Gly to Asp misincorporation in a peptide linker (such as e.g. a GS linker), said method comprising the step of replacing, in the nucleic acid sequence and/or nucleic acid that encodes said peptide linker, at least one GGC codon with a GGG or GGA.

In a further aspect, the invention provides a method for reducing the level of Gly to Asp misincorporation in a peptide linker (such as e.g. a GS linker) present in a multivalent (such as bivalent, trivalent, tetravalent) immunoglobulin single variable domain or Nanobody, said method comprising the step of replacing, in the nucleic acid sequence and/or nucleic acid that encodes said peptide linker, at least one GGC codon with a GGG, GGA or GGT/GGU codon.

In this aspect, the invention also provides a method for reducing the level of Gly to Asp misincorporation in a peptide linker (such as e.g. a GS linker) present in a multivalent (such as bivalent, trivalent, tetravalent) immunoglobulin single variable domain or Nanobody, said method comprising the step of replacing, in the nucleic acid sequence and/or nucleic acid that encodes said peptide linker, at least one GGC codon with a GGG or GGA.

The nucleotide sequences and nucleic acids described herein may be DNA or RNA (and are preferably double stranded DNA) and may be in the form of a genetic construct (for example in the form of a suitable vector, such as an expression vector). Such a genetic construct may for example, besides the nucleotide sequence encoding the (fusion) protein or polypeptide, comprise one or more suitable elements for expression of said nucleotide sequence, such as a suitable promoter, a suitable translation initiation sequence such as a ribosomal binding site and start codon, a suitable termination codon, and a suitable transcription termination sequence, 3′- or 5′-UTR sequences, leader sequences, selection markers, expression markers/reporter genes, and/or elements that may facilitate or increase (the efficiency of) transformation or integration, all suitably (and where appropriate, operably) linked to the nucleotide sequence encoding the (fusion) protein or polypeptide. Suitable examples of such elements will be clear to the skilled person and may for example depend upon the host or host cell in which said (expression) vector is to be expressed.

The genetic constructs described herein may also be in a form suitable for transformation of the intended host cell or host organism, in a form suitable for integration into the genomic DNA of the intended host cell or in a form suitable for independent replication, maintenance and/or inheritance in the intended host organism. For instance, the genetic constructs described herein may be in the form of a vector, such as for example a plasmid, cosmid, YAC, a viral vector or transposon. In particular, the vector may be an expression vector, i.e. a vector that can provide for expression in vitro and/or in vivo (e.g. in a suitable host cell, host organism and/or expression system). Such genetic constructs and (expression) vectors form further aspects of the invention.

Preferably, the regulatory and further elements of the genetic constructs described herein are such that they are capable of providing their intended biological function in the intended host cell or host organism.

For instance, a promoter, enhancer or terminator should be “operable” in the intended host cell or host organism, by which is meant that (for example) said promoter should be capable of initiating or otherwise controlling/regulating the transcription and/or the expression of a nucleotide sequence—e.g. a coding sequence—to which it is operably linked (as defined herein).

Some particularly preferred promoters include, but are not limited to, promoters known per se for the expression in the host cells mentioned herein; and in particular promoters for the expression in the bacterial cells, such as those mentioned herein.

A selection marker should be such that it allows—i.e. under appropriate selection conditions—host cells and/or host organisms that have been (successfully) transformed with a nucleotide sequence (as described herein) to be distinguished from host cells/organisms that have not been (successfully) transformed. Some preferred, but non-limiting examples of such markers are genes that provide resistance against antibiotics (such as kanamycin or ampicillin), genes that provide for temperature resistance, or genes that allow the host cell or host organism to be maintained in the absence of certain factors, compounds and/or (food) components in the medium that are essential for survival of the non-transformed cells or organisms.

A leader sequence should be such that—in the intended host cell or host organism—it allows for the desired post-translational modifications and/or such that it directs the transcribed mRNA to a desired part or organelle of a cell. A leader sequence may also allow for secretion of the expression product from said cell. As such, the leader sequence may be any pro-, pre-, or prepro-sequence operable in the host cell or host organism. Leader sequences may not be required for expression in a bacterial cell. For example, leader sequences known per se for the expression and production of antibodies and antibody fragments (including but not limited to single domain antibodies and ScFv fragments) may be used in an essentially analogous manner.

An expression marker or reporter gene should be such that—in the host cell or host organism—it allows for detection of the expression of (a gene or nucleotide sequence present on) the genetic construct. An expression marker may optionally also allow for the localisation of the expressed product, e.g. in a specific part or organelle of a cell and/or in (a) specific cell(s), tissue(s), organ(s) or part(s) of a multicellular organism. Such reporter genes may also be expressed as a protein fusion with the encoded amino acid sequence. Some preferred, but non-limiting examples include fluorescent proteins such as GFP.

Some preferred, but non-limiting examples of suitable promoters, terminator and further elements include those that can be used for the expression in the host cells mentioned herein; and in particular those that are suitable for expression in bacterial cells, such as those mentioned herein. For some (further) non-limiting examples of the promoters, selection markers, leader sequences, expression markers and further elements that may be present/used in the genetic constructs described herein—such as terminators, transcriptional and/or translational enhancers and/or integration factors—reference is made to the general handbooks such as Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987), as well as to the examples that are given in WO 95/07463, WO 96/23810, WO 95/07463, WO 95/21191, WO 97/11094, WO 97/42320, WO 98/06737, WO 98/21355, U.S. Pat. Nos. 7,207,410, 5,693,492 and EP 1 085 089. Other examples will be clear to the skilled person. Reference is also made to the general background art cited above and the further references cited herein.

Techniques for generating the nucleotide sequences, nucleic acids and genetic constructs described herein will be clear to the skilled person and may for instance include, but are not limited to, automated DNA synthesis. The genetic constructs described herein may also generally be provided by suitably linking the nucleotide sequence(s) described herein to the one or more further elements described above. Often, the genetic constructs described herein will be obtained by inserting a nucleotide sequence or nucleic acid as described herein in a suitable (expression) vector known per se. These and other techniques will be clear to the skilled person, and reference is again made to the standard handbooks, such as Sambrook et al. and Ausubel et al., mentioned above.

The nucleic acids described herein and/or the genetic constructs described herein may be used to transform a host cell or host organism, i.e. for expression and/or production of the encoded (fusion) protein or polypeptide. Suitable hosts or host cells will be clear to the skilled person, and may for example be any suitable fungal, prokaryotic or eukaryotic cell or cell line or any suitable fungal, prokaryotic or eukaryotic organism, for example:
a bacterial strain, including but not limited to gram-negative strains such as strains of Escherichia coli; of Proteus, for example of Proteus mirabilis; of Pseudomonas, for example of Pseudomonas fluorescens; and gram-positive strains such as strains of Bacillus, for example of Bacillus subtilis or of Bacillus brevis; of Streptomyces, for example of Streptomyces lividans; of Staphylococcus, for example of Staphylococcus carnosus; and of Lactococcus, for example of Lactococcus lactis;
a fungal cell, including but not limited to cells from species of Trichoderma, for example from Trichoderma reesei; of Neurospora, for example from Neurospora crassa; of Sordaria, for example from Sordaria macrospora; of Aspergillus, for example from Aspergillus niger or from Aspergillus sojae; or from other filamentous fungi;
a yeast cell, including but not limited to cells from species of Saccharomyces, for example of Saccharomyces cerevisiae; of Schizosaccharomyces, for example of Schizosaccharomyces pombe; of Pichia, for example of Pichia pastoris or of Pichia methanolica; of Hansenula, for example of Hansenula polymorpha; of Kluyveromyces, for example of Kluyveromyces lactis; of Arxula, for example of Arxula adeninivorans; of Yarrowia, for example of Yarrowia lipolytica;
an amphibian cell or cell line, such as Xenopus oocytes;
an insect-derived cell or cell line, such as cells/cell lines derived from lepidoptera, including but not limited to Spodoptera SF9 and Sf21 cells or cells/cell lines derived from Drosophila, such as Schneider and Kc cells;
a plant or plant cell, for example in tobacco plants; and/or
a mammalian cell or cell line, for example a cell or cell line derived from a human, a cell or a cell line from mammals including but not limited to CHO-cells, BHK-cells (for example BHK-21 cells) and human cells or cell lines such as HeLa, COS (for example COS-7) and PER.C6 cells;
   as well as all other hosts or host cells known per se for the expression and production of antibodies and antibody fragments (including but not limited to (single) domain antibodies and ScFv fragments), which will be clear to the skilled person. Reference is also made to the general background art cited hereinabove, as well as to for example WO 94/29457; WO 96/34103; WO 99/42077; Frenken et al. (1998, Res. Immunol. 149(6): 589-99); Riechmann and Muyldermans (1999, J. Immunol. Methods, 231(1-2): 25-38); van der Linden (2000, J. Biotechnol. 80(3): 261-70); Joosten et al. (2003, Microb. Cell Fact. 2(1): 1); Joosten et al. (2005, Appl. Microbiol. Biotechnol. 66(4): 384-92); and the further references cited herein.

Some preferred expression hosts are Pichia pastoris and human cell lines used for the expression/production of therapeutic proteins.

The term “GS linkers” as used herein generally refers to peptide linkers that are comprised of and/or essentially consist of glycine and serine residues.

Generally, such GS linkers (as well as other peptide linkers referred to herein) will contain at least 5 amino acid residues, such as about 10 amino acid residues, about 15 amino acid residues, about 20 amino acid residues, about 25 amino acid residues, about 35 amino acid residues, and up to 50 amino acid residues or more (although usually, linkers comprising about 10 to 40 amino acid residues, such as about 15 to about 35 amino acid residues, will often be used in practice).

Usually, such linkers will contain an excess of glycine residues compared to the number of serine residues, for example between 3 and 6 glycine residues for each serine residue. Usually also, such linkers will contain one or more (such as two or more) repeats of a sequence motif. Also, although in the invention in its broadest sense, the presence of one or more other amino acids (such as a glutamic acid residue, or a threonine residue instead of a serine residue) is not excluded, the linkers used herein preferably only contain (or are intended to only contain) glycine and serine residues.

As will be clear to the skilled person, the GS linkers that are most commonly used in the art of protein engineering (and which are also preferred in the practice of the present invention) are linkers that comprise one or more repeats of the GGGGS (SEQ ID NO: 1) motif, i.e. linkers of the general formula (Gly-Gly-Gly-Gly-Ser)ₙ, in which n may be 1, 2, 3, 4, 5, 6, 7 or more. Some examples as 15GS linkers (n=3) and 35GS linkers (n=7). Reference is for example made to Chen et al., Adv Drug Deliv. Rev. 2013 Oct. 15; 65(10): 1357-1369; and Klein et al., Protein Eng. Des. Sel. (2014) 27 (10): 325-330.

The GS linkers encoded by the GS linker-encoding sequence(s) of the invention can be used to link together, in a suitable manner, any desired proteins, peptides, peptide moieties, binding domains or binding units, so as to form a (fusion) protein or polypeptide in which two or more of such proteins, peptides, peptide moieties, binding domains or binding units are linked together by one or more GS linkers. Generally, and as will be clear to the skilled person, the GS linkers encoded by the GS linker-encoding sequence(s) of the invention can be used for any purpose for which GS linkers can be used and/or have been used in the prior art. Such uses and applications of the GS linker-encoding sequence(s) of the invention (and of the GS linkers encoded by the same) will be clear to the skilled person.

In one specific aspect, the GS linkers encoded by the GS linker-encoding sequence(s) of the invention can suitably be used to link together two or more immunoglobulin single variable domains (such as two or more Nanobodies, e.g. VHH's, humanized VHH's, sequence-optimized VHH's, or camelized VH's, such as camelized human VH's), to form bivalent, trivalent, bispecific, trispecific, biparatopic, tetravalent, or other suitable ISVD constructs. Reference is for example made to the various applications by Ablynx N.V., such as for example and without limitation WO 2004/062551, WO 2006/122825, WO 2008/020079 and WO 2009/068627. The GS linkers may for example also be used to link one or more immunoglobulin single variable domains or Nanobodies against a therapeutic target to an immunoglobulin single variable domain or Nanobody that provides for increased half-life (e.g. increased t1/2-beta), such as an immunoglobulin single variable domain or Nanobody against serum albumin. Again, in these uses or applications, the GS linker-encoding sequence(s) of the invention (and GS linkers encoded by the same) can be used in essentially the same way as known nucleotide sequences that encode GS linkers. Some specific but non-limiting examples of such immunoglobulin single variable domain or Nanobody constructs are schematically shown in Table III, and nucleic acids encoding these constructs are also schematically shown in Figure I (the legend of Table III applies). Other examples will be clear to the skilled person based on the disclosure herein.

TABLE III

| | Structure |
|---|---|
| Type of Nanobody construct | (schematically represented) |
| Bivalent, monospecific | Nb₁-L₁-Nb₁ |
| Bivalent, bispecific | Nb₁-L₁-Nb₂ |
| Bivalent, biparatopic | Nb_{A}-L₁-Nb_{B} |
| Trivalent, monospecific | Nb₁-L₁-Nb₁-L₂-Nb₁ |
| Trivalent, bispecific | Nb₁-L₁-Nb₁-L₂-Nb₂ |
| Trivalent, bispecifie | Nb₁-L₁-Nb₂-L₂-Nb₁ |
| Trivalent, bispecific | Nb₂-L₁-Nb₁-L₂-Nb₁ |
| Trivalent, trispecifie | Nb₁-L₁-Nb₂-L₂-Nb₃ |
| Trivalent, biparatopic | Nb_{A}-L₁-Nb_{B}-L₂-Nb₂ |

“Nb₁” refers to a first Nanobody.
“Nb₂” refers to a second Nanobody, binding to a target different from the target that Nb₁ binds to (Nb₂ may for example also bind to serum albumin in order to confer improved half-life).
“Nb_{A}” and “Nb_{B}”, respectively, refer to a first and second Nanobody binding to different epitopes on the same target.
“L₁” refers to a first GS linker
“L₂” refers to a second GS linker, which may or may not be the same as L₁

The invention will now be further described by means of the following non-limiting preferred aspects, examples and figures, in which:

### FIGURE LEGENDS

FIG. 1 schematically shows some non-limiting examples of Nanobody constructs containing linkers;

FIG. 2 schematically shows the tetravalent Nanobody construct used in Example 1 to illustrate the invention. FIG. 2 also shows the localization of the T10 peptide in this construct;

FIG. 3 shows the amino acid sequence (SEQ ID NO:10) and codon usage (SEQ ID NO: 11) of peptide T10. In the sequence, amino acid residues and codons where a misincorporation with aspartic acid was observed are indicated in bold/underline (note, for the residues/codons indicated in italics/underline, misincorporation could have been expected but was not observed).

FIG. 4 shows the amino acid sequence (SEQ ID NO:12) and coding sequences (SEQ ID NOs: 13 to 15) of the 35 GS linkers in Nanobody Construct A. Specific codons for glycine susceptible for misincorporation with aspartic acid (GGT and GGC) are indicated in bold/underline. Codons for serine are annotated in small caps.

FIG. 5 shows a cation exchange chromatogram of purified Nanobody Construct A on Source 15S column (GE Healthcare Life Sciences) and a pH gradient (green trace, CX-1 pH gradient buffer A (pH 5.6) and B (pH 10.2), Thermo Scientific), recorded at UV 254 nm (red (lower) trace) and UV 280 nm (blue (upper) trace). pH recording is shown in gray trace. The pre-peaks are acidic variants of Nanobody Construct A. The fractions 14, 15, 16, and 17 were pooled for subsequent characterization of the acidic variants, and fraction 18 for characterization of the main peak;

FIG. 6 shows the Max-ent deconvoluted mass spectra obtained for acidic variants (top pane) and main peak (bottom pane) collected from cation exchange fractionation of purified Nanobody Construct A. The most important mass measured in the acidic fractions is 59689.4 Da, which is 58 Dalton higher than the mass of Nanobody Construct A as measured in the pH-IEX main peak fraction (59630.9 Da, see bottom pane);

FIG. 7 lists the peptide fragments (SEQ ID NOs: 16 to 33) of tryptic peptide T10 generated by an Asp-N digest, an endoproteinase cleaving at the N-terminus of an aspartic acid. Each cleavage site corresponds with a glycine exchanged to an aspartic acid;

FIG. 8 shows the relative levels of Gly to Asp misincorporation of three sites (C1, C2, and C3) in the GS linker(s) of (a) Nanobody construct A; (b) Nanobody construct A after depletion of variants with Asp misincorporation by pH-IEX; (c) Nanobody construct A in which 100% of GGC codon sequences were replaced with a GGG, GGA or GGT codon sequence;

FIG. 9 shows the ten constructs that were produced to investigate the impact of valency and linker length on Gly to Asp misincorporation as described in Example 3;

FIG. 10 shows the relative levels of Gly to Asp misincorporation of the two sites (C1 and C2) in the 9GS linker; (A) bivalent construct, (B) trivalent construct, (C) tetravalent construct;

FIG. 11 shows the relative levels of Gly to Asp misincorporation of the five sites (C1, C2, C3, C4, and C5) in the 20GS linker; (A) bivalent construct, (B) trivalent construct, (C) tetravalent construct;

FIG. 12 shows the relative levels of Gly to Asp misincorporation of the nine sites (C1 to C9) in the 35GS linker; (A) bivalent construct, (B) trivalent construct and (C) tetravalent construct, (D) tetravalent construct without GGC codons.

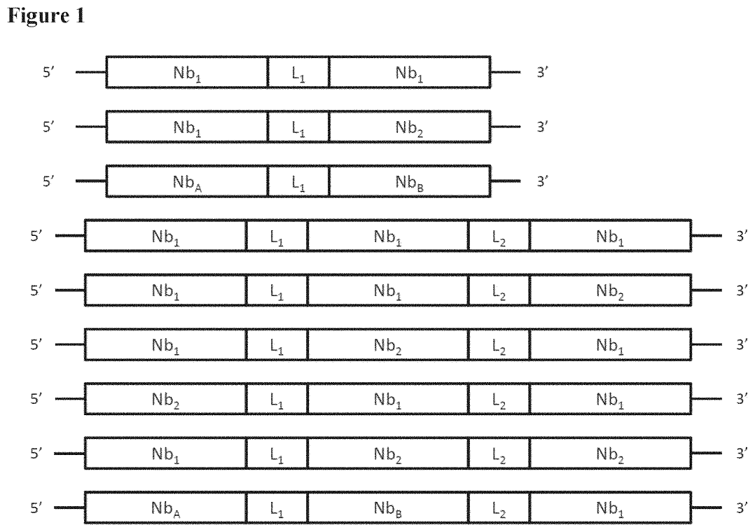

The entire contents of all of the references (including literature references, issued patents, published patent applications, and co pending patent applications) cited throughout this application are hereby expressly incorporated by reference, in particular for the teaching that is referenced hereinabove.

### EXPERIMENTAL PART

### Example 1

### Construction of an Expression Vector for a Tetravalent Nanobody Construct

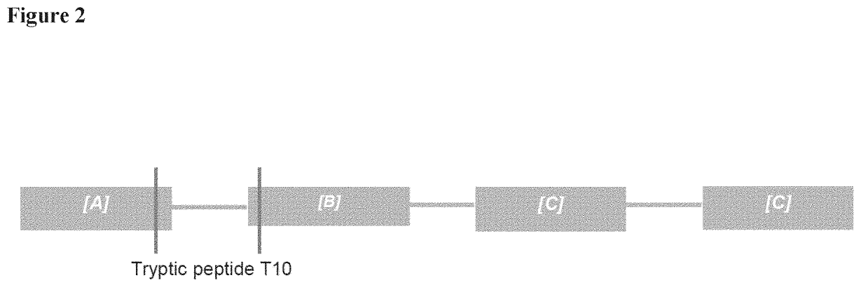

In this Example, the invention will be illustrated using, as a non-limiting example, a tetravalent Nanobody construct consisting of four sequence optimized variable domains of a heavy-chain llama antibody, which are fused head-to-tail with 35GS linkers (see FIG. 2). The overall construct used (also referred to herein as “Nanobody construct A”) can be schematically represented by the formula

[A]-[35GS linker]-[B]-[35GS linker]-[C]-[35GS linker]-[C]

in which [A], [B] and [C] represent three different Nanobodies and [35GS linker] represents a 35GS linker (see also FIG. 2).

DNA fragments containing the coding information of Nanobody Construct A were cloned into the multiple cloning site of a Pichia expression vector that contains a Zeocin™ resistance gene (a derivative of the original pPpT4_Alpha_S expression vector described by Näätsaari et al., PLoS One. 2012; 7(6):e39720), such that the Nanobody® sequence was downstream of and in frame with the alfa Mating Factor (aMF) signal peptide sequence.

Transformation of the Nanobody Construct a Coding Sequence, Expression and Secretion of the Construct in Pichia pastoris

Transformation and expression studies were performed in the Pichia strain NRRL Y-11430 (ARS Patent Culture Collection 1815 North University St., Peoria). This WT strain was used to make a derivative strain overexpressing the endogenous Pichia auxiliary protein KAR2 (GeneID:8198455) as well as Nanobody Construct A. Both Nanobody Construct A and Kar2 were under the control of the AOX1 methanol inducible promoter. Transformation was performed by standard techniques and in accordance with the standard handbooks (see for example Methods In Molecular Biology 2007, Humana Press Inc.). Transformants were grown on selective medium containing Zeocin and a number of individual colonies were selected and evaluated on the expression level of Nanobody Construct A in 5 mL shake-flasks cultures in BMCM medium and induced by the addition of methanol as has been described in Pichia protocols (see again the standard handbooks). The best expressing clone was used in standard fed batch fermentation. Glycerol fed batches were performed and induction was initiated by the addition of methanol. The productions were performed at 2 L scale at pH6, 30° C. in complex medium with a methanol feed rate of 4 ml/L*h.

Purification of the Nanobody Construct a after Fed-Batch Fermentation

Nanobody Construct A was purified as follows: after fermentation, part of the cell broth was clarified via a hollow fiber 750 kDa followed by a capture step using a CIEX Poros XS resin, a polish step using CIEX Nuvia HR-S resin and a flow through step on an AIEX Sartobind STIC PA. Finally a concentration and buffer exchange step was performed via UF/DF using the Hydrosart 10 kD membrane.

### Analysis of Purified Nanobody Construct a on Ion Exchange Chromatography and Determination of Molecular Weight of Acidic Variants

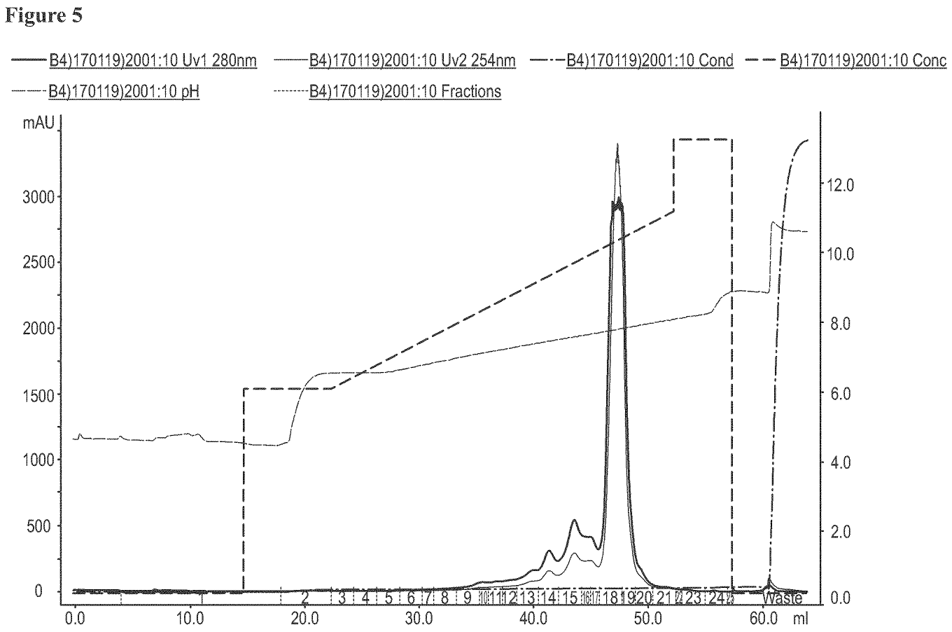

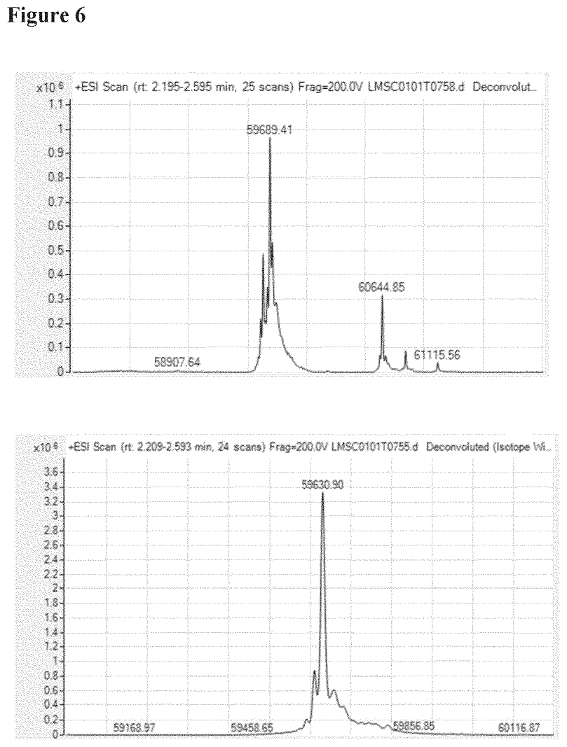

The purified Nanobody Construct A was analyzed by strong cation exchange chromatography using a pH gradient (pH-IEX). The chromatogram, shown in FIG. 5, shows acidic variants of the Nanobody® A eluting as a group of pre-peaks relative to the main peak. After fraction collection of the acidic and main peaks, the nature of the acidic variants was investigated by determining their molecular weight by electrospray Q-TOF mass spectrometry. The deconvoluted mass spectra are shown in FIG. 6. The main mass observed in the acidic fraction was 59689.4 Da, which is 58 Dalton higher than the mass of Nanobody Construct A as measured in the pH-IEX main peak fraction. The mass measured for Nanobody Construct A in the main peak fraction (59630.9 Da) is 12 ppm higher than theoretical molecular weight of Nanobody Construct A, i.e. within the measurement error of the instrument.

A 58 Dalton mass difference can be explained by the exchange of glycine with the acidic amino acid aspartic acid.

Analysis and Identification of Acidic Variants by Peptide Map Reversed Phase UHPLC Coupled with Mass Spectrometry (RP-UHPLC-MS)

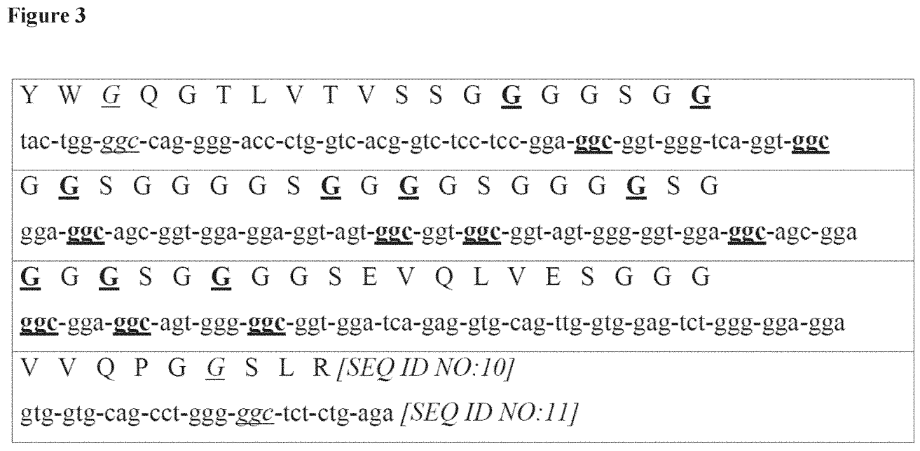

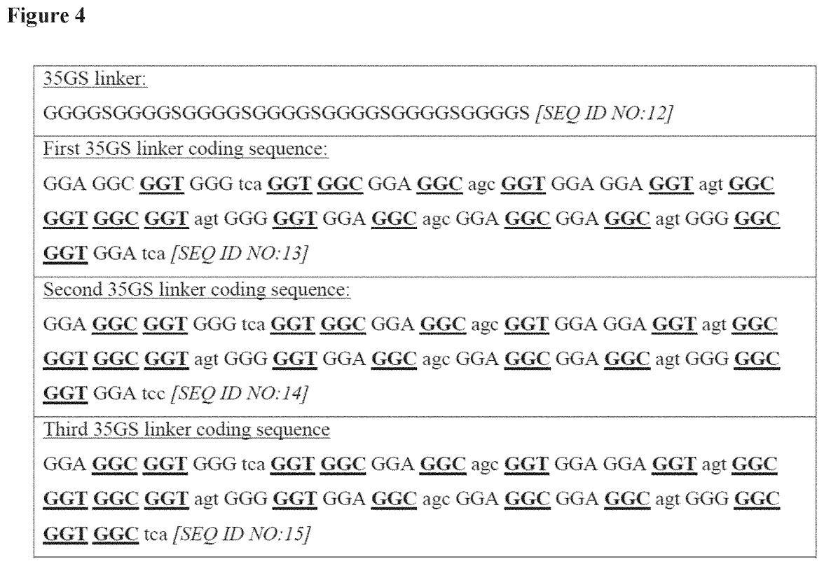

Peptide map analysis (after trypsin digest) of the acidic variants fraction of Nanobody Construct A resulted in identification of two peptides with a mass increment of 58 Dalton. As schematically shown in FIG. 2, one of these two peptides (referred to herein as the “T10 peptide”) corresponds to a part of the sequence that encompasses a few of the C-terminal amino acid residues of the first Nanobody in the construct, the first 35Gs linker and a few of the N-terminal amino acid residues of the second Nanobody in the construct. The amino acid sequence (SEQ ID NO:10) and nucleotide sequence (SEQ ID NO:11) of the T10 peptide are shown in FIG. 3.

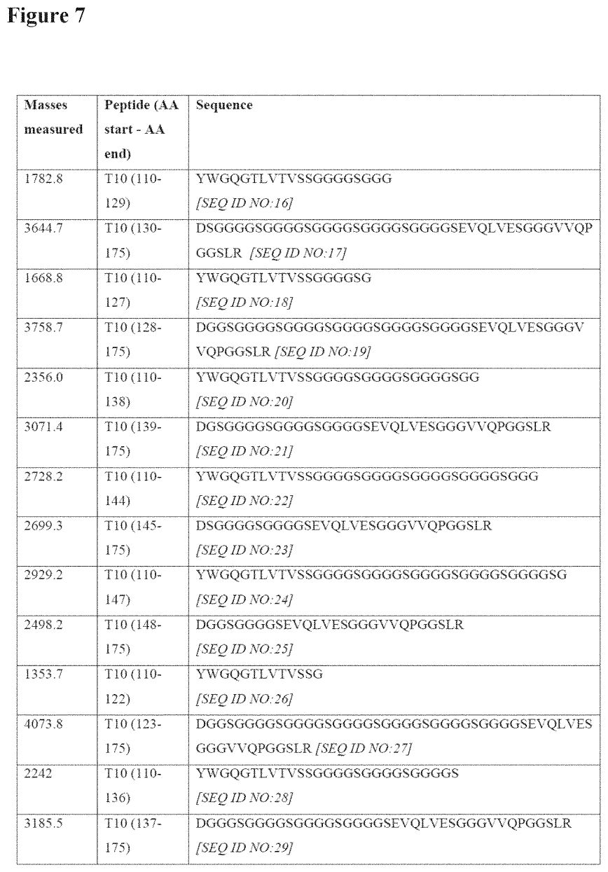

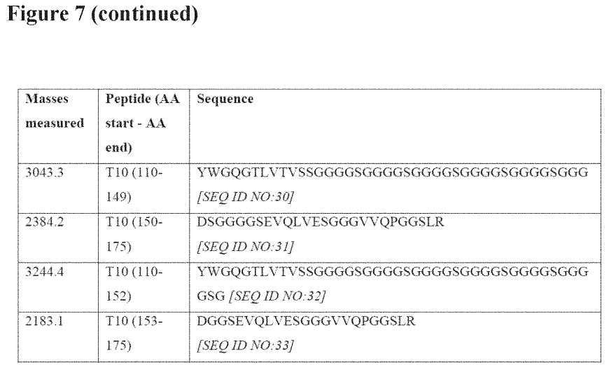

As collision induced fragmentation in the mass spectrometer led to only partial sequence coverage of the T10 peptide, the T10 peptide of the trypsin digest was fractionated by reversed phase chromatography, and subsequently digested with the enzyme Asp-N. The enzyme Asp-N is an endoproteinase that hydrolyses peptide bonds on the N-terminal side of aspartic acid residues. Because no aspartic acid residues are in the sequence of this peptide, cleavages were only expected in case of a Gly->Asp misincorporation events. In the analysis of the Asp-N digest of the T10 peptide by RP-UHPLC-MS, different fragments were identified with a mass corresponding to fragments of the T10 peptide with a mass increment of 58 Dalton. In total 9 Asp-N fragmentation sites were identified, as shown in FIG. 7. Quite unexpectedly, it was observed that the Asp misincorporation only occurred at GGC codons (see also FIG. 3), and not at GGT codons although both glycine codons can in principle be misread by the aspartic acid tRNAs (having the anticodons CUG and CUA). In both cases there is a G-(mRNA)/U-(tRNA) mismatch, i.e. the most common mismatch during translation, along with wobble position mismatches (C/U and/or U/U), that cause amino acid misincorporation. Thus, more generally, according to the invention, when a codon encoding glycine other than GGA or GGG (i.e. that is not GGA or GGG) is present in a nucleotide sequence of the invention, it may be preferred that codon is GGT or GGU rather than GGC.

As mentioned, the peptide map analysis of Nanobody Construct A also resulted in identification of a second peptide with a mass increment of 58 Dalton. This peptide was found to correspond to one of the CDR's of one of the Nanobodies present in Nanobody Construct A. Further analysis (data not shown) confirmed that also for this peptide, the observed mass increment of 58 Dalton was most likely due to Asp misincorporation.

### Example 2: Codon Optimization in the Nucleic Acid Sequence of the 35GS Linkers

The GGC codon sequences present in the 35GS linker sequence of Nanobody construct A were replaced with a GGG, GGA or GGT codon sequence.

The obtained Nanobody constructs were expressed in Pichia strain NRRL Y-11430 and purified as described above. The level of Asp misincorporation in the obtained polypeptides was measured by the same method as described above. The mass spectrometer was setup to quantify 3 out of 9 misincorporation sites.

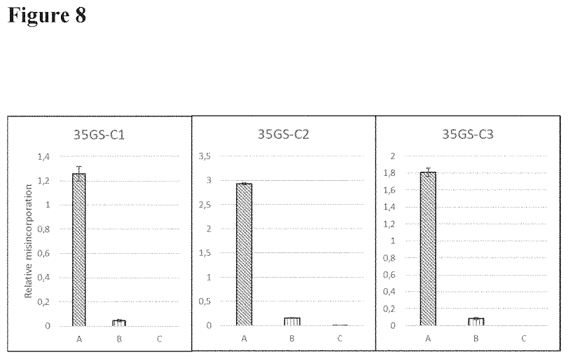

The relative levels of Asp misincorporation in the 35GS linker of the polypeptide obtained with the Reference Nanobody construct A (no codon optimization) and of the polypeptide obtained with the codon optimized Nanobody construct A is shown in FIG. 8.

### Example 3: Observation of Asp Misincorporation in Other Linkers

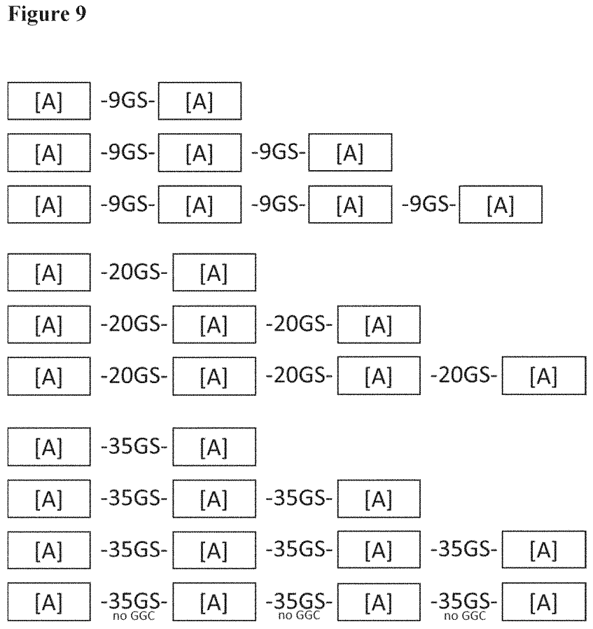

In this example, the impact of Nanobody valency and linker length on Gly to Asp misincorporation was studied. For this, bi-, tri- and tetravalent constructs, each with 9GS, 20GS or 35GS linkers sequences and a Nanobody building block sequence (different from the Nanobody building block sequence present in Nanobody construct A) were produced. An extra tetravalent, 35GS linker Nanobody construct was also produced without any GGC codons. The ten new constructs are shown in FIG. 9. The 9GS linker contains 2 GGC codons, the 20GS linker contains 5 GGC codons and the 35GS linker contains 9 GGC codons.

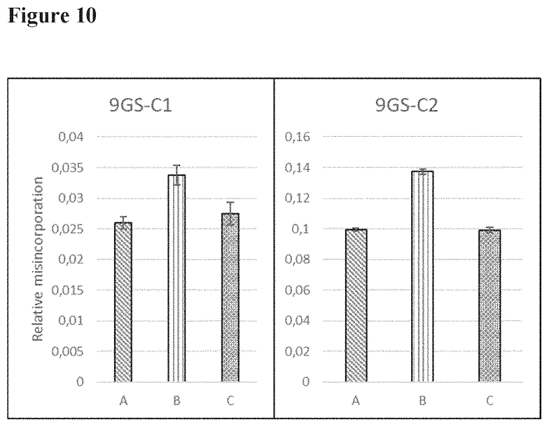

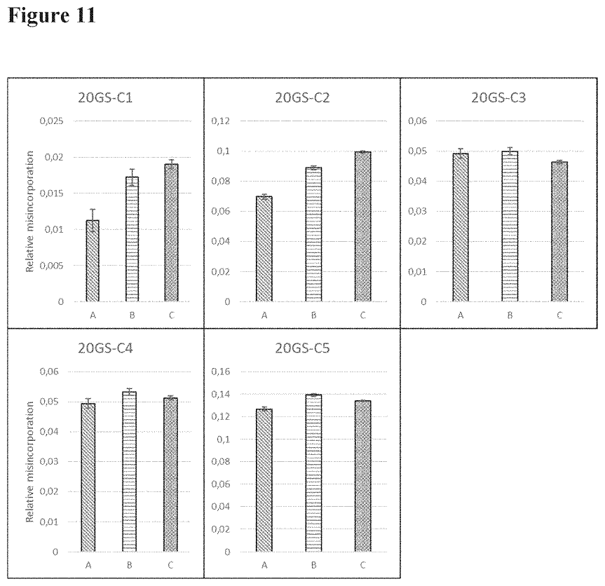

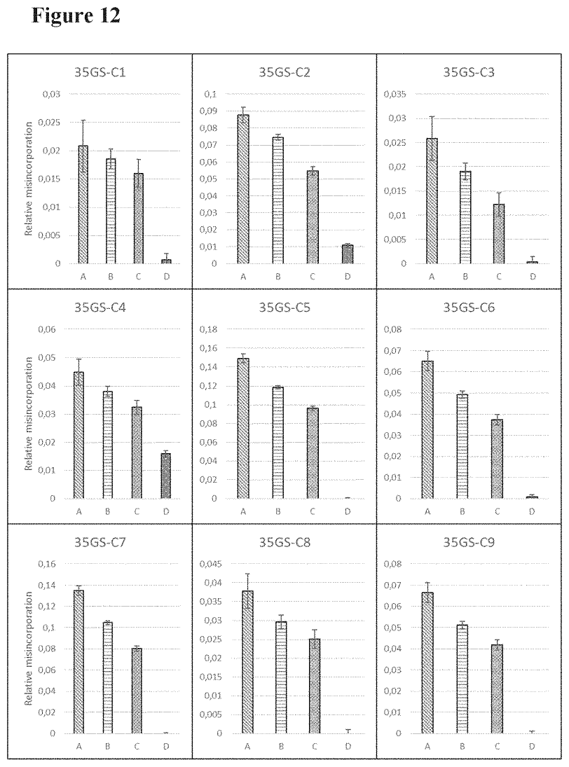

Each possible new peptide after Gly to Asp misincorporation was followed with the mass spectrometry method as described above. The method was further optimized to allow simultaneous quantification of all 9 Asp-N fragmentation sites. The results on the misincorporation are shown in FIG. 10 (9GS linker), FIG. 11 (20 GS linker) and FIG. 12 (35 GS linker).

From these results it can be concluded that the valency or the linker length does not have an impact on Gly to Asp misincorporation levels. Removal or reduction of the number of GGC codons clearly reduces the level of Gly to Asp misincorporation.

Finally, although the invention is described herein mainly with respect to GS linkers, it will be clear to the skilled person that the invention can generally be applied to other peptide linkers that contain glycine residues.

Thus, in a further aspect, the invention relates to a nucleotide sequence and/or a nucleic acid that encodes a peptide linker, in which the peptide linker encoded by nucleotide sequence and/or a nucleic acid contains four or more glycine residues, in which more than 70%, preferably more than 85%, more preferably more than 90%, such as more than 95% and up to 99% and more (including 100%) of the codons that encode a glycine residue in the GS linker are either GGA, GGG or GGT/GGU.

In this aspect, the invention also relates to a nucleotide sequence and/or a nucleic acid that encodes a peptide linker, in which the peptide linker encoded by nucleotide sequence and/or a nucleic acid contains four or more glycine residues, in which more than 70%, preferably more than 85%, more preferably more than 90%, such as more than 95% and up to 99% and more (including 100%) of the codons that encode a glycine residue in the GS linker are either GGA or GGG.

In this aspect, the invention also relates to a nucleotide sequence and/or a nucleic acid that encodes a peptide linker, in which the peptide linker encoded by nucleotide sequence and/or a nucleic acid contains four or more glycine residues, in which less than 30%, preferably less than 1%, more preferably less than 10%, such as less than 5% and up to less than 1% and lower (including 0%) of the codons that encode a glycine residue in the GS linker are GGC.

1. Nucleotide sequence and/or a nucleic acid that encodes a peptide linker, in which the peptide linker encoded by said nucleotide sequence or nucleic acid comprises or (essentially) consists of glycine and serine residues, in which:
more than 70%, preferably more than 85%, more preferably more than 90%, such as more than 95% and up to 99% and more (including 100%) of the codons that encode a glycine residue in said peptide linker are either GGA, GGG, or GGT/GGU;
more than 70%, preferably more than 85%, more preferably more than 90%, such as more than 95% and up to 99% and more (including 100%) of the codons that encode a glycine residue in said peptide linker are either GGA or GGG; and/or
less than 30%, preferably less than 15%, more preferably less than 10%, such as less than 5% and up less than 1% and lower (including 0%) of the codons that encode a glycine residue in said peptide linker are GGC.

2. Nucleotide sequence and/or a nucleic acid according to claim 1, in which more than 70%, preferably more than 85%, more preferably more than 90%, such as more than 95% and up to 99% or more (including 100%) of the codons that encode a glycine residue in said peptide linker are either GGA, GGG, or GGT/GGU.

3. Nucleotide sequence and/or a nucleic acid according to any of claim 1 or 2, in which more than 70%, preferably more than 85%, more preferably more than 90%, such as more than 95% and up to 99% or more (including 100%) of the codons that encode a glycine residue in said peptide linker are either GGA or GGG.

4. Nucleotide sequence and/or a nucleic acid according to any of claims 1 to 3, in which less than 30%, preferably less than 15%, more preferably less than 10%, such as less than 5% and up less than 1% or lower (including 0%) of the codons that encode a glycine residue in said peptide linker are GGC.

5. Nucleotide sequence and/or a nucleic acid according to any of claims 1 to 4, in which said peptide linker comprises or (essentially) consists of one or more (such as two or more) repeats of the sequence motif GGGGS (SEQ ID NO:1).

6. Nucleotide sequence and/or a nucleic acid according to any of claims 1 to 5, in which said peptide linker is a 9 GS linker, a 15 GS linker, a 20 GS linker, or a 35 GS linker.

7. Nucleotide sequence and/or a nucleic acid according to claim 6, in which said peptide linker is a 35 GS linker.

8. Nucleotide sequence and/or a nucleic acid that encodes a (fusion) protein or fusion polypeptide, in which the fusion protein or polypeptide that is encoded by said nucleotide sequence and/or a nucleic acid comprises two or more peptide moieties that are suitably linked via one or more peptide linkers, in which said one or more peptide linkers are encoded by a nucleotide sequence or nucleic acid according to any of claims 1 to 7.

9. Nucleotide sequence and/or a nucleic acid according to claim 8, in which the two or more peptide moieties are both immunoglobulin single variable domains.

10. Nucleotide sequence and/or a nucleic acid according to claim 9, in which the two or more peptide moieties are both VHH's, humanized VHH's, sequence-optimized VHH's, or camelized VH's, such as camelized human VH's.

11. Nucleotide sequence and/or a nucleic acid according to any of claims 8 to 10, which encodes a bivalent, trivalent, bispecific, trispecific, biparatopic, or tetravalent construct.

12. Genetic construct that comprises a nucleotide sequence and/or a nucleic acid according to any of claims 1 to 11.

13. Method for expressing or producing a (fusion) protein or polypeptide, in which said method at least comprises the step of expressing a nucleotide sequence or nucleic acid according to any of claims 8 to 11 in a suitable host cell or host organism, and optionally also comprises the step of isolating/purifying the (fusion) protein or polypeptide thus expressed.

14. Method for expressing or producing a (fusion) protein or polypeptide according to claim 12, wherein the host is Pichia, such as Pichia pastoris.

15. Method for expressing or producing a (fusion) protein or polypeptide according to claim 12, wherein the host is a mammalian cell, such as a Chinese hamster ovary (CHO) cell.

16. A host cell or host organism that comprises a nucleotide sequence and/or a nucleic acid that encodes a (fusion) protein or fusion polypeptide according to any of claims 8 to 11.

17. Method for reducing the level of Gly to Asp misincorporation in a peptide linker, said method comprising the step of replacing, in the nucleic acid sequence and/or nucleic acid that encodes said peptide linker, at least one GGC codon with a GGG, GGA or GGT/GGU codon.

18. Method for reducing the level of Gly to Asp misincorporation in a peptide linker according to claim 17, wherein the at least one GGC codon is replaced with a GGG or GGA codon.

19. Method for reducing the level of Gly to Asp misincorporation in a peptide linker according to any of claim 17 or 18, wherein the peptide linker comprises or (essentially) consists of one or more (such as two or more) repeats of the sequence motif GGGGS (SEQ ID NO:1).

20. Method for reducing the level of Gly to Asp misincorporation in a peptide linker according to any of claims 17 to 19, wherein the peptide linker is a 9 GS linker, a 15 GS linker, a 20 GS linker, or a 35 GS linker.

21. Method for reducing the level of Gly to Asp misincorporation in a peptide linker according to any of claims 17 to 20, wherein the peptide linker is a 35 GS linker.

22. Method for reducing the level of Gly to Asp misincorporation in a peptide linker according to any of claims 17 to 21, wherein the peptide linker links two or more peptide moieties.

23. Method for reducing the level of Gly to Asp misincorporation in a peptide linker according to claim 22, wherein the peptide moieties are immunoglobulin single variable domains.

24. Method for reducing the level of Gly to Asp misincorporation in a peptide linker according to claim 23, wherein the peptide moieties are VHH's, humanized VHH's, sequence-optimized VHH's, or camelized VH's, such as camelized human VH's.

25. Method for reducing the level of Gly to Asp misincorporation in a peptide linker according to any of claims 22-24, wherein the peptide linker is comprised in a bivalent, trivalent, bispecific, trispecific, biparatopic, or tetravalent construct.
